(19) United States
(12) Patent Application Publication
(10) Pub. No.: US 2023/0375540 A1
(43) Pub. Date: Nov. 23, 2023

(54) Protein Damage in Aging and Age-Related Diseases

(71) Applicant: Mediterranean Institute for Life Sciences, Split (HR)

(72) Inventors: Miroslav Radman, Hvar (HR); Anita Krisko, Varazdin (HR)

(21) Appl. No.: 18/052,492

(22) Filed: Nov. 3, 2022

Related U.S. Application Data
(63) Continuation of application No. 16/371,432, filed on Apr. 1, 2019, now abandoned.
Application No. 16/371,432 is a continuation of application No. 15/699,664, filed on Sep. 8, 2017, now abandoned.
Application No. 15/699,664 is a continuation of application No. 14/181,661, filed on Feb. 15, 2014, now abandoned.
(60) Provisional application No. 61/765,370, filed on Feb. 15, 2013.

(51) Int. Cl. G01N 33/543; G01N 33/68; G01N 33/84

(52) U.S. Cl. CPC G01N 33/54306; G01N 33/6893; G01N 33/84; G01N 2800/2821; G01N 2800/2835

(57) ABSTRACT

The process of aging and the development of age-related diseases are related to the emerging phenotypes of increasingly damaged and progressively malfunctioning proteomes. The present invention provides methods of preventing aging and age-related diseases in mammals by assessment of protein-specific oxidative damage. Methods of providing treatments that reduce intracellular reactive oxygen and/or nitrogen species, or protein-specific damage caused by reactive oxygen and/or nitrogen species, are disclosed. Furthermore, methods of screening for compounds that reduce intracellular reactive oxidative species, and/or molecules that prevent protein-specific damage by protecting the susceptible protein from such damage and therefore prevent or treat degenerative or age-related diseases, are also disclosed.

### CROSS REFERENCES

This application claims the benefit of U.S. Provisional Application Ser. No. 61/765,370, filed Feb. 15, 2013, which is incorporated herein by reference in its entirety.

### FIELD OF THE INVENTION

The present invention, in some embodiments, concerns methods of diagnosing the cause of, and/or treating, a degenerative disease, an age-related disease, or a progeroid syndrome, and methods of preventing aging and age-related diseases in mammals including the steps of assessment of protein-specific oxidative damage and providing a treatment that reduces protein-specific oxidative damage. In other embodiments, the present invention also relates to monitoring protein-specific oxidative damage and providing treatments that reduce intracellular reactive oxygen species, and to methods of screening for compounds that reduce intracellular reactive oxidative species, reduce protein-specific damage, and prevent degenerative diseases.

### BACKGROUND OF THE INVENTION

Aging is the accelerated degradation of cellular activities causing progressive malfunction, morbidity and death. T. Kirkwood wrote that “aging is arguably the most familiar yet least well-understood aspect of human biology” [1]. The coexistence of hundreds of theories of aging [1] demonstrates that the lack of a fundamental concept is a bottleneck in productive research on aging. For instance, nearly all of the current research on aging deals with the “downstream” consequences of aging rather than with fundamental process of aging, called intrinsic aging [1], and its cause(s). Because of the complexity of the manifestations of aging, there is a generally accepted opinion is that aging and its causes are very complicated. However, the complexity of the consequences of aging reflects the complexity of the organism, not that of the cause of aging.

Substantial evidence shows that irreversible oxidative proteome damage (protein carbonylation, PC), of the kind that accumulates exponentially with human or animal age [2], leads to increasing cellular malfunction, morbidity and mortality [3-5].

All phenotypes, including morbidity and mortality, are due to change in protein function that can occur directly at protein level without genomic change. Destroying protein function via gene mutation is basically an irreversible event, whereas the phenotype of direct protein damage can be reversible as long as the gene remains intact. It is the proteome that sustains life, whereas genome assures its perpetuation by the ongoing renewal of the proteome (granted the capacity of that proteome to repair, replicate and express the genome). Although proteome damage increases mutation rates [5], L. Orgel's error catastrophe scenario [6] is conceivable also solely at the proteome level (e.g., via damage to proteome protection, repair and turnover systems) without the necessary involvement of DNA mutations, with the notable exception of cancer.

### SUMMARY OF THE INVENTION

In an embodiment, disclosed herein is a method of screening for therapies for a disease in a mammal including the steps of: (1) performing a diagnostic measurement of protein-specific oxidative damage in a protein related to the disease and (2) screening for a preventive method or composition and/or a treatment of the disease that reduces the level of the monitored protein-specific oxidative damage.

In an embodiment, disclosed herein is a method of screening for therapies for a disease in a mammal including the steps of: (1) performing a measurement of protein-specific oxidative damage in a protein and (2) screening for a preventive method or composition and/or a treatment of the disease that reduces the level of protein-specific oxidative damage, wherein the disease is selected from the group consisting of an age-related disease, a degenerative disease, or a progeroid syndrome.

In an embodiment, disclosed herein is a method of screening for therapies for a disease in a mammal comprising the steps of: (1) performing a measurement of protein-specific oxidative damage in a protein and (2) screening for a preventive method or composition and/or a treatment of the disease by reducing the level of protein-specific oxidative damage, wherein the measurement of protein-specific oxidative damage is a measurement of protein carbonylation.

In an embodiment, disclosed herein is a method of screening for preventative methods, compositions, or therapies for a disease in a mammal including the steps of: (1) performing a measurement of protein-specific oxidative damage in a protein and (2) screening for a preventive method or composition and/or a treatment of the disease that reduces the level of protein-specific oxidative damage, wherein the treatment reduces at least one intracellular reactive oxygen species.

In an embodiment, disclosed herein is a method of screening for preventative methods, compositions, or therapies for a disease in a mammal including the steps of: (1) performing a measurement of protein-specific oxidative damage in a protein and (2) screening for a preventive method or composition and/or a treatment of the disease that reduces the level of protein-specific oxidative damage, wherein the treatment reduces at least one intracellular reactive nitrogen species.

In an embodiment, disclosed herein is a method of screening for preventative methods, compositions, or therapies for a disease in a mammal including the steps of: (1) performing a measurement of protein-specific oxidative damage in a protein and (2) screening for a preventive method or composition and/or a treatment of the disease that reduces the level of protein-specific oxidative damage, wherein the treatment reduces the susceptibility of the protein to oxidative damage.

In an embodiment, disclosed herein is a method of preventing or treating a disease in a mammal including the steps of (1) performing a diagnostic measurement of protein-specific oxidative damage in a protein and (2) providing a treatment to the mammal.

In an embodiment, disclosed herein is a method of preventing or treating a disease in a mammal including the steps of (1) performing a diagnostic measurement of protein-specific oxidative damage in a protein and (2) providing a treatment to the mammal, wherein the treatment reduces at least one intracellular reactive oxygen species.

In an embodiment, disclosed herein is a method of preventing or treating a disease in a mammal including the steps of (1) performing a diagnostic measurement of protein-specific oxidative damage in a protein and (2) providing a treatment to the mammal, wherein the treatment reduces at least one intracellular reactive nitrogen species.

In an embodiment, disclosed herein is a method of preventing or treating a disease in a mammal including the steps of (1) performing a diagnostic measurement of protein-specific oxidative damage in a protein and (2) providing a treatment to the mammal, wherein the treatment reduces protein susceptibility to oxidative damage.

### BRIEF DESCRIPTION OF THE DRAWINGS

The foregoing summary, as well as the following detailed description of the invention, will be better understood when read in conjunction with the appended drawings.

FIG. 1 illustrates protein carbonyl content from different species and tissues versus biological time (fraction of lifespan).

FIG. 2 illustrates experimental results showing that single amino acid substitutions alter the susceptibility of Parkinson's disease-associated α-synuclein to oxidative damage induced by γ-rays.

FIG. 3 shows an expanded region of the data shown in FIG. 2, highlighting the effects of lower γ-ray irradiation doses.

### DETAILED DESCRIPTION OF THE INVENTION

Statistically, the incidences of all age-related diseases and death increase exponentially with age and display similar slopes (Gompertz curves) hinting at a common underlying species-specific somatic biological clock. Based on published work [2-5], we propose that the chemistry of this biological clock is oxidative protein damage that, as it accumulates with age, causes an avalanche of morbid phenotypes resulting from expanding cellular malfunctions that eventually affect entire organs and organisms.

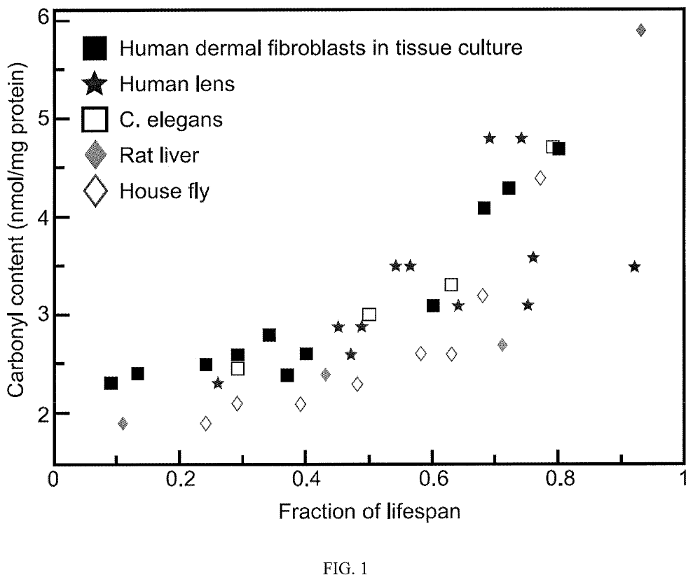

We suggest that the process of aging has a simple cause with complex consequences and propose a basic concept that aging and age-related diseases are the emerging phenotypes of the accumulating proteome damage. We present here a simple hypothesis that can interpret observations concerning aging.

Whereas the exponential increase in the accumulation of cellular PC within the life span of several very different species (from nematode to human) is remarkably similar (FIG. 1) [2], the chronological time (from three weeks to hundred years) is characteristic of the species. However, when E. Stadtman and colleagues developed the method for PC measurement and observed the exponential relationship with age, they could not know whether PC is just a parallel of aging, its consequence, or cause. PC appears as the best single biomarker of aging that was used over 25 years simply as a quantitative indicator of oxidative stress. Now, we have arguments to think that oxidative proteome damage is the probable fundamental cause of aging and age-related diseases [5].

The changing steady-state level of oxidative proteome damage depends on: (i) the rate of production of free radicals—e.g. highly reactive oxygen and nitrogen species (ROS, RNS, etc. . . . ), (ii) the efficacy of scavenging and enzymatic detoxification of, and protection against, free radicals, as well as the efficacy of molecular repair of incurred oxidative damage and the turnover of damaged molecules [7], and (iii) the degree of intrinsic resistance to oxidative damage of each individual protein species. Individual proteins show great inequalities in their susceptibility to oxidative damage: the majority of native proteins are resilient to oxidative damage [8, 9]. But, such evolved protein resistance to oxidation is fragile because it is readily lost by errors in amino acid sequence [5, 10] or mistakes in folding [5, 8]. This observation will become interesting when considering the molecular basis of inter-individual differences of the biological effects of advanced age.

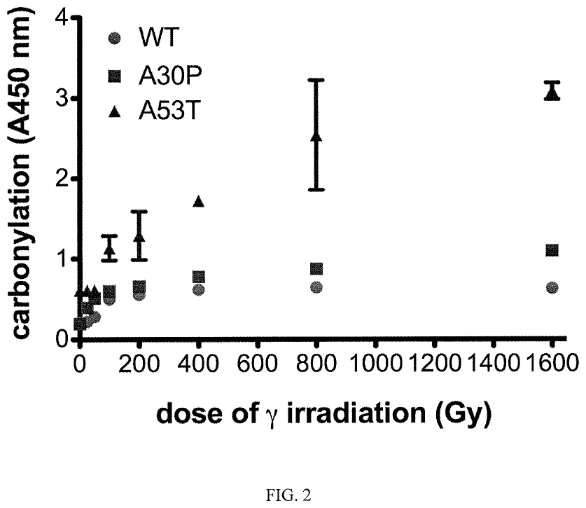

We posit that inter-individual differences in the kind and time of an outcome of aging (e.g., an age-related disease) could be accounted for by the remarkable effect of protein sequence variation (i.e., the “neutral” polymorphism by single amino acid substitutions) on the variation in protein susceptibility to oxidative damage (FIG. 2 and legend). It is likely that—at equal ROS levels—some individual proteins will oxidize in vivo at different rates in different individuals of the same age and health status. However, at an advanced age, because of that difference in protein oxidation, carriers of a more oxidation-sensitive protein may predictably suffer from a particular age-related disease. The underlying concept is that proteome polymorphism, functionally neutral at young age, becomes progressively phenotypic at advanced ages. This is a hypothesis for a potential future predictive diagnostics of a person's predisposition to some age-related disease, based on oxy-proteomics—a kind of personalized bio-medical “profiling” of the human population.

Of all age-related degenerative diseases, only cancer seems to require genomic alterations (mutation in specific genes and/or specific chromosomal rearrangements) to lock-in the functional change bestowing stable malignant phenotype. Cancer originates in very rare single cells acquiring a “dominant” trait (unlike cell malfunction or death) such as “immortality” by the loss of apoptosis and the acquisition of a loss of contact inhibition leading to an unlimited cell division potential. Even then, the cause of mutation is likely due to the defect in the part of the proteome that is responsible for the maintenance of DNA sequence [5, 11] and structural genome integrity, be it nuclear or mitochondrial.

All other degenerative diseases must originate in a functional defect of a large fraction of cells of an entire organ or tissue. It cannot yet be excluded that rare dominant negative mutations prevail in aging of most of somatic cells, it is unlikely that organs fail, and organisms age, because majority of cells acquire recessive somatic mutations in both copies of the same gene, such as in hereditary syndromes. There is no hint of such massive mutagenesis in centenarian genome studies. Furthermore, rejuvenation of the cells of centenarians by the passage through the highly proliferative iPS stage, followed by re-differentiation [12], shows that: (i) neither nuclear nor mitochondrial genomes undergo a massive mutagenesis that could account for irreversible malfunction and morbidity of all aging cells, and (ii) there is no other fatally irreversible change in all age-affected cells that can prevent cell rejuvenation by protein turnover.

There is ample evidence that a degenerative chronic disease (e.g., Alzheimer's disease or cancer) presumably commences stochastically with a small number of defective cells usually a decade, or rather decades, before an avalanche-like process of acquired malfunctions affects the majority of cells constituting the diseased organ. This malfunction probably originates from the low amount or low quality of some members of the cellular proteome. In the genesis of cancer, proteome damage can cause damage to the genome (mutations), as seen in bacteria [5], and conceivably also reduce the lag in phenotypic expression of emerging homozygous recessive mutations (particularly in quiescent stem cells) by destroying, e.g., by oxidative damage, the residual activity of the remaining protein. Protein dilution by successive cell divisions would have the same effect. This could account for, or contribute to, tumor promotion by chronic inflammation.

As an example of the cause-consequence conundrum, telomerase activity can decrease because of damage to telomerase itself (or to other members of the complex) or to the proteins required for the expression of the relevant gene. Inflammation is a recurrent source of oxidative proteome damage with diverse deleterious biological consequences. In general, it is difficult to imagine a physiological change unrelated to a significant change in protein performance. A “null” gene mutation is the source of the most severe protein deficiency but, unless inherited, it is too infrequent to handicap the function of an entire organ during aging. However, a direct damage (e.g., oxidative) to the relevant protein, or damage to the proteins involved in its synthesis, folding, modification or turnover, is initially less severe than null mutation but it can become increasingly severe with time and affect all cells of an organ. Therefore such phenotypic change will require time for protein damage to accumulate before causing a functional deficiency. Such a scenario is compatible with the phenomenology of aging.

This proteome-based hypothesis of the intrinsic aging process can formally interpret the complexity of manifestations of aging and provide guidelines for testing its veracity. We elaborate and specify the original general theory of aging by oxidative stress [13] by taking into account limited but significant results obtained with a wide variety of cells, from bacterial to human [3-12]. The elaborated concept offers a new framework for studying the origin of degenerative diseases ravaging the oldest part of the human population.

Because most proteins are resilient to oxidation [8, 9], only rare proteins becoming susceptible to oxidative damage by hereditary or acquired mutation, or perhaps by post-synthetic modification, or lack of it, require protection from such damage. Gene mutation or direct damage to proteins that control proteome quality (e.g., chaperones, proteasome and autophagosome) may accelerate aging by increasing the sensitivity to oxidation of a large fraction of the proteome. Proteins assuring the functionality of mitochondria and the cytoplasmic redox potential can affect cellular ROS levels and therefore the amount of damage to oxidation-susceptible proteins.

If this concept of aging turns out to be correct, then measuring total PC and protein-specific oxidative damage (a diagnostic of predisposition), and searching for effective protection against it (prevention and therapy), could become a public health priority. Negative results of some clinical trials with single antioxidants can not be taken into account at this point because: (i) no single antioxidant is known to neutralize a dozen of ROS and RNS varieties and (ii) no clinical trial with standard antioxidants showed that the treatment was effective in reducing intracellular ROS and PC. Novel potent antioxidant activities have surfaced recently: the most effective proteome protective antioxidant metabolite mixtures and, to a lesser extent, the increased known detoxifying enzymatic activities [6] accounting for extreme biological robustness have been recently found in cells of the most resilient organisms [3, 4, 14].

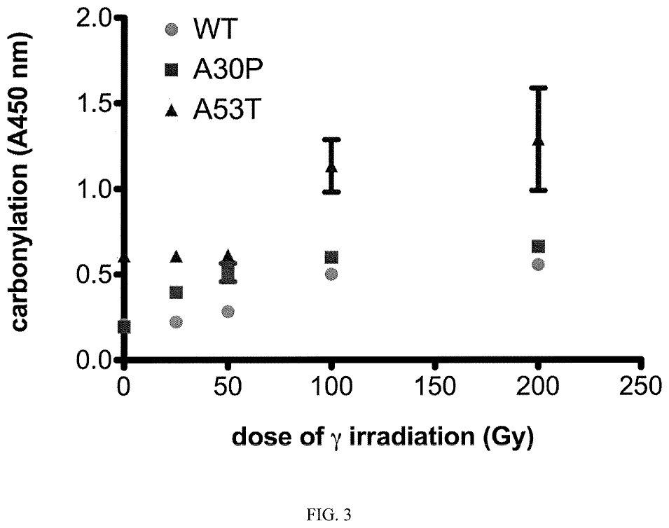

The hypothesis that age-related diseases originate from increased cellular PC receives support also from the findings that, with exception of immortalized cancer cells, all tested degenerative diseases are associated with increased PC [15]. Yet, increased oxidizability of one or few protein species is not necessarily expected to increase the overall cellular PC levels. Therefore, the validation of the proposed concept will require systematic studies of many personal oxy-proteomes (e.g., 2-dimensional electrophoretic “carbonylomes”) to identify proteins with increased carbonylation (as in FIGS. 2 and 3) within recognizable pathways of specific age-related diseases. If successful, such studies would mark the beginning of a new predictive and symptomatic diagnostics of everybody's predisposition to some age-related disease (i.e., the inborn weakest link) and allow the development of personalized preventive and curative medicine by the use of effective antioxidants. Whatever the chance of success, an eventual active prevention of chronic degenerative diseases would provide for a revolution in public health comparable only with prevention of infectious diseases by vaccination.

A degenerative disease, as defined herein, is a disease that is characterized by progressive degenerative changes in tissue. Degenerative diseases include, but are not limited to, Alzheimer's disease, Parkinson's disease, heart disease, osteoarthritis, osteoporosis, Huntington's disease, type II diabetes, cancer, and neurodegenerative diseases. For example, the involvement of oxidative stress in neurodegenerative disorders is well-recognized [18-26]. In an embodiment, a degenerative disease is selected from the group consisting of Alzheimer's disease, Parkinson's disease, heart disease, osteoarthritis, osteoporosis, Huntington's disease, type II diabetes, cancer, and neurodegenerative diseases.

An age-related disease, as defined herein, is a disease that is characterized by progressive increases in symptoms as the biological age of a patient or subject increases. Age-related diseases generally occur with increased frequency with increasing senescence. Age-related diseases include, but are not limited to, macular degeneration, age-related macular degeneration, cataracts, glaucoma, diabetic retinopathy, Alzheimer's disease, senile dementia, type II diabetes, arthritis, osteoarthritis, osteoporosis, cancer, cardiovascular disease (e.g. aneurysm, angina, strokes, and hypertension), Parkinson's disease, depression, erectile dysfunction, hearing loss, bladder incontinence, and chronic obstructive pulmonary disease. In an embodiment, an age-related disease is selected from the group consisting of macular degeneration, age-related macular degeneration, cataracts, glaucoma, diabetic retinopathy, Alzheimer's disease, senile dementia, type II diabetes, arthritis, osteoarthritis, osteoporosis, cancer, cardiovascular disease (e.g. aneurysm, angina, strokes, hypertension), Parkinson's disease, depression, erectile dysfunction, hearing loss, bladder incontinence, and chronic obstructive pulmonary disease.

A progeroid syndrome, as defined herein, is a rare disorder that mimics physiological aging. Progeroid syndromes include, but are not limited to, disorders that arise from defects in DNA repair (e.g. Werner syndrome, Bloom syndrome, Cockayne syndrome, xeroderma pigmentosum, trichothiodystrophy), disorders that arise from defects in lamin A/C (e.g. Hutchinson-Gilford progeria syndrome, restrictive dermopathy), and disorders with unknown causes (e.g. Wiedemann-Rautenstrauch syndrome). In an embodiment, a progeroid syndrome is selected from the group consisting of disorders that arise from defects in DNA repair, disorders that arise from defects in lamin A/C, and disorders with unknown causes. In another embodiment, a progeroid syndrome is selected from the group consisting of Werner syndrome, Bloom syndrome, Cockayne syndrome, xeroderma pigmentosum, trichothiodystrophy, Hutchinson-Gilford progeria syndrome, restrictive dermopathy, and Wiedemann-Rautenstrauch syndrome.

In an embodiment, disclosed herein is a method of screening for therapies for a disease in a mammal including the steps of: (1) performing a measurement of protein-specific oxidative damage in a protein and (2) screening for a treatment of the disease that reduces the level of protein-specific oxidative damage.

In an embodiment, disclosed herein is a method of screening for therapies for a disease in a mammal including the steps of: (1) performing a measurement of protein-specific oxidative damage in a protein and (2) screening for a treatment of the disease that reduces the level of protein-specific oxidative damage, wherein the disease is selected from the group consisting of an age-related disease, a degenerative disease, or a progeroid syndrome.

In an embodiment, disclosed herein is a method of screening for therapies for a disease in a mammal including the steps of: (1) performing a measurement of protein-specific oxidative damage in a protein and (2) screening for a treatment of the disease that reduces the level of protein-specific oxidative damage, wherein the measurement of protein-specific oxidative damage is a phenotypic screen.

In an embodiment, disclosed herein is a method of screening for therapies for a disease in a mammal including the steps of: (1) performing a measurement of protein-specific oxidative damage in a protein and (2) screening for a treatment of the disease that reduces the level of protein-specific oxidative damage, wherein the measurement of protein-specific oxidative damage is a measurement of protein carbonylation.

In an embodiment, disclosed herein is a method of screening for therapies for a disease in a mammal including the steps of: (1) performing a measurement of protein-specific oxidative damage in a protein and (2) screening for a treatment of the disease that reduces the level of protein-specific oxidative damage, wherein the treatment reduces at least one intracellular reactive oxygen species.

In an embodiment, disclosed herein is a method of screening for therapies for a disease in a mammal including the steps of: (1) performing a measurement of protein-specific oxidative damage in a protein and (2) screening for a treatment of the disease that reduces the level of protein-specific oxidative damage, wherein the treatment reduces at least one intracellular reactive nitrogen species.

In an embodiment, disclosed herein is a method of screening for therapies for a disease in a mammal including the steps of: (1) performing a measurement of protein-specific oxidative damage in a protein and (2) screening for a treatment of the disease that reduces the level of protein-specific oxidative damage, wherein the treatment reduces the susceptibility of the protein to oxidative damage.

In an embodiment, disclosed herein is a method of screening for therapies for a disease in a mammal including the steps of: (1) performing a measurement of protein-specific oxidative damage in a protein and (2) screening for a treatment of the disease that reduces the level of protein-specific oxidative damage, wherein the protein is α-synuclein.

In an embodiment, disclosed herein is a method of screening for therapies for a disease in a mammal including the steps of: (1) performing a measurement of protein-specific oxidative damage in a protein and (2) screening for a treatment of the disease that reduces the level of protein-specific oxidative damage, wherein the disease is selected from the group consisting of an age-related disease, a degenerative disease, or a progeroid syndrome, wherein the protein is α-synuclein.

In an embodiment, disclosed herein is a method of screening for therapies for a disease in a mammal including the steps of: (1) performing a measurement of protein-specific oxidative damage in a protein and (2) screening for a treatment of the disease that reduces the level of protein-specific oxidative damage, wherein the measurement of protein-specific oxidative damage is a phenotypic screen, wherein the protein is α-synuclein.

In an embodiment, disclosed herein is a method of screening for therapies for a disease in a mammal including the steps of: (1) performing a measurement of protein-specific oxidative damage in a protein and (2) screening for a treatment of the disease that reduces the level of protein-specific oxidative damage, wherein the measurement of protein-specific oxidative damage is a measurement of protein carbonylation, wherein the protein is α-synuclein.

In an embodiment, disclosed herein is a method of screening for therapies for a disease in a mammal including the steps of: (1) performing a measurement of protein-specific oxidative damage in a protein and (2) screening for a treatment of the disease that reduces the level of protein-specific oxidative damage, wherein the treatment reduces at least one intracellular reactive oxygen species, wherein the protein is α-synuclein.

In an embodiment, disclosed herein is a method of screening for therapies for a disease in a mammal including the steps of: (1) performing a measurement of protein-specific oxidative damage in a protein and (2) screening for a treatment of the disease that reduces the level of protein-specific oxidative damage, wherein the treatment reduces at least one intracellular reactive nitrogen species, wherein the protein is α-synuclein.

In an embodiment, disclosed herein is a method of screening for therapies for a disease in a mammal including the steps of: (1) performing a measurement of protein-specific oxidative damage in a protein and (2) screening for a treatment of the disease that reduces the level of protein-specific oxidative damage, wherein the treatment reduces the susceptibility of the protein to oxidative damage, wherein the protein is α-synuclein.

In an embodiment, disclosed herein is a method of treating a disease in a mammal including the steps of (1) performing a diagnostic measurement of protein-specific oxidative damage in a protein and (2) providing a treatment to the mammal.

In an embodiment, disclosed herein is a method of treating a disease in a mammal including the steps of (1) performing a diagnostic measurement of protein-specific oxidative damage in a protein and (2) providing a treatment to the mammal, wherein the treatment reduces at least one intracellular reactive oxygen species.

In an embodiment, disclosed herein is a method of treating a disease in a mammal including the steps of (1) performing a diagnostic measurement of protein-specific oxidative damage in a protein and (2) providing a treatment to the mammal, wherein the treatment reduces at least one intracellular reactive nitrogen species.

In an embodiment, disclosed herein is a method of treating a disease in a mammal including the steps of (1) performing a diagnostic measurement of protein-specific oxidative damage in a protein and (2) providing a treatment to the mammal, wherein the treatment reduces protein susceptibility to oxidative damage.

In an embodiment, disclosed herein is a method of treating a disease in a mammal including the steps of (1) performing a diagnostic measurement of protein-specific oxidative damage in a protein and (2) providing a treatment to the mammal, wherein the treatment reduces at least one intracellular reactive oxygen species and reduces protein susceptibility to oxidative damage.

In an embodiment, disclosed herein is a method of treating a disease in a mammal including the steps of (1) performing a diagnostic measurement of protein-specific oxidative damage in a protein and (2) providing a treatment to the mammal, wherein the treatment reduces at least one intracellular reactive nitrogen species and reduces protein susceptibility to oxidative damage.

In an embodiment, disclosed herein is a method of treating a disease in a mammal including the steps of (1) performing a diagnostic measurement of protein-specific oxidative damage in a protein and (2) providing a treatment to the mammal, wherein the treatment reduces at least one intracellular reactive oxidative species, reduces at least one reactive nitrogen species, and reduces protein susceptibility to oxidative damage.

In an embodiment, disclosed herein is a method of treating a disease in a mammal including the steps of (1) performing a diagnostic measurement of protein-specific oxidative damage in a protein and (2) providing a treatment to the mammal, wherein the disease is selected from the group consisting of an age-related disease, a degenerative disease, or a progeroid syndrome.

In an embodiment, disclosed herein is a method of treating a disease in a mammal including the steps of (1) performing a diagnostic measurement of protein-specific oxidative damage in a protein and (2) providing a treatment to the mammal, wherein the diagnostic measurement of protein-specific oxidative damage is a phenotypic screen.

In an embodiment, disclosed herein is a method of treating a disease in a mammal including the steps of (1) performing a diagnostic measurement of protein-specific oxidative damage in a protein and (2) providing a treatment to the mammal, wherein the diagnostic measurement of protein-specific oxidative damage is a measurement of protein carbonylation.

In an embodiment, disclosed herein is a method of treating a disease in a mammal including the steps of (1) performing a diagnostic measurement of protein-specific oxidative damage in a protein and (2) providing a treatment to the mammal, wherein the protein is α-synuclein.

In an embodiment, disclosed herein is a method of treating a disease in a mammal including the steps of (1) performing a diagnostic measurement of protein-specific oxidative damage in a protein and (2) providing a treatment to the mammal, wherein the treatment reduces at least one intracellular reactive oxygen species, wherein the protein is α-synuclein.

In an embodiment, disclosed herein is a method of treating a disease in a mammal including the steps of (1) performing a diagnostic measurement of protein-specific oxidative damage in a protein and (2) providing a treatment to the mammal, wherein the treatment reduces at least one intracellular reactive nitrogen species, wherein the protein is α-synuclein.

In an embodiment, disclosed herein is a method of treating a disease in a mammal including the steps of (1) performing a diagnostic measurement of protein-specific oxidative damage in a protein and (2) providing a treatment to the mammal, wherein the treatment reduces protein susceptibility to oxidative damage, wherein the protein is α-synuclein.

In an embodiment, disclosed herein is a method of treating a disease in a mammal including the steps of (1) performing a diagnostic measurement of protein-specific oxidative damage in a protein and (2) providing a treatment to the mammal, wherein the treatment reduces at least one intracellular reactive oxygen species and reduces protein susceptibility to oxidative damage, wherein the protein is α-synuclein.

In an embodiment, disclosed herein is a method of treating a disease in a mammal including the steps of (1) performing a diagnostic measurement of protein-specific oxidative damage in a protein and (2) providing a treatment to the mammal, wherein the treatment reduces at least one intracellular reactive nitrogen species and reduces protein susceptibility to oxidative damage, wherein the protein is α-synuclein.

In an embodiment, disclosed herein is a method of treating a disease in a mammal including the steps of (1) performing a diagnostic measurement of protein-specific oxidative damage in a protein and (2) providing a treatment to the mammal, wherein the treatment reduces at least one intracellular reactive oxidative species, reduces at least one reactive nitrogen species, and reduces protein susceptibility to oxidative damage, wherein the protein is α-synuclein.

In an embodiment, disclosed herein is a method of treating a disease in a mammal including the steps of (1) performing a diagnostic measurement of protein-specific oxidative damage in a protein and (2) providing a treatment to the mammal, wherein the disease is selected from the group consisting of an age-related disease, a degenerative disease, or a progeroid syndrome, wherein the protein is α-synuclein.

In an embodiment, disclosed herein is a method of treating a disease in a mammal including the steps of (1) performing a diagnostic measurement of protein-specific oxidative damage in a protein and (2) providing a treatment to the mammal, wherein the diagnostic measurement of protein-specific oxidative damage is a phenotypic screen, wherein the protein is α-synuclein.

In an embodiment, disclosed herein is a method of treating a disease in a mammal including the steps of (1) performing a diagnostic measurement of protein-specific oxidative damage in a protein and (2) providing a treatment to the mammal, wherein the diagnostic measurement of protein-specific oxidative damage is a measurement of protein carbonylation, wherein the protein is α-synuclein.

The invention is further described by the following non-limiting example.

### EXAMPLE

### Example 1

This example shows that mistakes in amino acid sequence lead to increased oxidative susceptibility. The example chosen for this study is the protein α-synuclein, which is a protein found in the brain that is associated with Parkinson's disease. Two point mutations associated with Parkinson's disease in the gene coding for α-synuclein, A30P and A53T, are studied here in comparison to wild type (WT) α-synuclein [16, 17].

Samples of α-synuclein, wild type and two mutants (A3OP and A53T) were purchased from rPeptide (Bogart, GA, USA). Proteins were dissolved in 10 mM phosphate-buffered saline (PBS) with pH 7.4 at a final concentration of 1 mg/mL.

In order to prepare proteins for irradiation, samples were diluted by using 10 mM PBS pH 7.4 to final concentration of 0.01 mg/mL. A volume of 1 mL of each sample was irradiated in Eppendorf tubes on ice using ¹²⁶Cs as a source of gamma radiation to final doses of 50, 100, 200, 400, 800 and 1600 Gy. Immediately after irradiation, 100 μL of each sample was loaded on NUNC MAXISORP wells (Thermo Fisher Scientific, Pittsburgh, PA, USA) and incubated overnight at 4° C. to allow proteins to adsorb to the surface prior to their carbonylation measurement. Adsorbed proteins were derivatized by using 12 μg/mL dinitrophenylhydrazine (DNPH). Derivatization of adsorbed proteins is followed by detection of derivatized dinitrophenol (DNP)-carbonyl by a rabbit anti-DNP primary antibody (D9656, Sigma-Aldrich Co., St. Louis, MO, USA) and goat anti-rabbit secondary antibody conjugated to horseradish peroxidase (HRP) (111-035-14, Jackson ImmunoResearch Laboratories, Inc., Newmarket, Suffolk, UK). Stocks of antibodies were prepared at −1 μg/μL and used at 1:7000 dilutions. Subsequent incubation with enzyme substrate 3,3′,5,5′-tetramethylbenzidine resulted in a colored product that was quantified using a microplate reader with maximum absorbance at 450 nm.

The results of the experiments are shown in FIGS. 2 and 3. In FIG. 2, the single amino acid substitutions are seen to alter the susceptibility of α-synuclein to oxidative damage induced by γ-rays. FIG. 3 shows an expanded region of the data in FIG. 2 to highlight the effects of lower doses of γ-rays. The A30T and A53T substitutions show significantly increased protein carbonylation in comparison to WT α-synuclein. The results demonstrate that protein misfolding leads to an increased susceptibility to protein carbonylation.

### REFERENCES

1. T. B. L. Kirkwood, Understanding the odd science of aging. Cell 120, 437-447 (2005).
2. C. N. Oliver, B. W. Ahn, E. J. Moerman, S. Goldstein, E. R. Stadtman, Age-related changes in oxidized proteins. J. Biol. Chem. 262, 5488-5491 (1987).
3. A. Krisko, M. Radman, Protein damage and death by radiation in Escherichia coli and Deinococcus radiodurans. Proc. Natl. Acad. Sci. USA, 107, 14373-14377 (2010).
4. A. Krisko, M. Leroy, M. Radman, M. Meselson, Extreme anti-oxidant protection against ionizing radiation in bdelloid rotifers. Proc. Natl. Acad. Sci. USA 109, 2354-2357 (2012).
5. A. Krisko, M. Radman, Phenotypic and genetic consequences of protein damage. PLOS Genetics, 9, e1003810 (2013).
6. L. E. Orgel, The maintenance of the accuracy of protein synthesis and its relevance to ageing. Proc. Natl. Acad. Sci. USA 49, 517-521 (1963).
7. D. Slade, M. Radman, Oxidative stress resistance in Deinococcus radiodurans. Microbial. Mol. Biol. Rev. 75, 133-191 (2011).
8. A. Fredriksson, M. Ballesteros, S. Dukan, T. Nystrom, Defense against protein carbonylation by DnaK/DnaJ and proteases of the heat shock regulon. J. Bacterial. 187, 4207-4213 (2005).
9. E. K. Ahmed, A. Rogowska-Wrzesinska, P. Poepstorff, A.-L. Bulteau, B. Friguet, Protein modification and replicative senescence of WI-38 human embryonic fibroblasts. Aging Cell 9, 252-272 (2010).
10. S. Dukan, A. Farewell, M. Ballesteros, F. Taddei, M. Radman, T. Nystrom, Protein oxidation in response to increased transcriptional or translational errors. Proc. Natl. Acad. Sci. USA 97, 5746-5749 (2000).
11. M. M. Slupska, C. Baikalov, R. Lloyd, J. H. Miller, Mutator tRNAs are encoded by the Escherichia coli mutator genes mutA and mutC: A novel pathway for mutagenesis. Proc. Acad. Nat. Sci. USA 93, 4380-4385 (1996).
12. L. Lapasset, O. Milhavet, A. Prieur et al. Rejuvanating senescent and centenarian human cells by reprogramming through the pluripotent state. Genes and Dev. 25, 2248-2253 (2011).
13. D. Harman, Aging: A theory based on free radical and radiation chemistry. J. Gerontal. 11, 298-300 (1956).
14. M. J. Daly, E. K. Gaidamakova, V. Y. Matrosova, J. G. Kiang, R. Fukumoto, et al., Small-molecule antioxidant proteome-shields in Deinococcus radiodurans. PLOS One 5, e12570 (2010).
15. I. Dalle-Donne, D. Giustarini, R. Colombo, R. Rossi, A. Milzani, Protein carbonylation in human diseases. Trends Mol. Med. 9, 169-176 (2003).
16. M. H. Polymeropoulos C. Lavedan, E. Leroy, S. E. Ide, A. Dehejia, A. Dutra, B. Pike, H. Root, J. Rubenstein, R. Boyer, E. S. Stenroos, S. Chandrasekharappa, A. Athanassiadou, T. Papapetropoulos, W. G. Johnson, A. M. Lazzarini, R. C. Duvoisin, G. Di Iorio, L. I. Golbe, R. L. Nussbaum, Mutation in the alpha-synuclein gene identified in families with Parkinson's disease. Science 276, 2045-2047 (1997).
17. R. Krüger, W. Kuhn, T. Müller, D. Woitalla, M. Graeber, S. Kösel, H. Przuntek, J. T. Epplen, L. Schöls, O. Riess, Ala30Pro mutation in the gene encoding alpha-synuclein in Parkinson's disease. Nat. Genet. 18, 106-108 (1998).
18. K. Jomova, D. Vondrakova, M. Lawson, M. Valko, Metals, oxidative stress and neurodegenerative disorders. Mol. Chem. Biochem. 345, 91-104 (2010).
19. K. G. Manton, S. Volovik, A. Kulminski, ROS effects on neurodegeneration in Alzheimer's disease and related disorders: on environmental stresses of ionizing radiation. Curr. Alzheimer Res. 1, 277-293 (2004).
20. J. Y. Wang, L. L. Wen, Y. N. Huang, Y. T. Chen, M. C. Ku, Dual effects of antioxidants in neurodegeneration: direct neuroprotection against oxidative stress and indirect protection via suppression of glia-mediated inflammation. Curr. Pharm. Des. 12, 3521-3533 (2006).
21. L. H. Sanders, T. Greenamyre, Oxidative damage to macromolecules in human Parkinson disease and the rotenone model. Free Radic. Biol. Med. 62, 111-120 (2013).
22. A. Vaccaro, S. A. Patten, D. Aggad, et al: Pharmacological reduction of ER stress protects against TDP-43 neuronal toxicity in vivo. Neurobiol. Dis. 55, 64-75 (2013).
23. F. Morroni, A. Tarozzi, G. Sita, et al., Neuroprotective effect of sulforaphane in 6-hydroxydopamine-lesioned mouse model of Parkinson's disease. Neurotoxicology, 36, 63-71 (2013).
24. R. Li, N. Zheng, T. Liang, Q. He, L. Xu, Puerarin attenuates neuronal degeneration and blocks oxidative stress to elicit a neuroprotective effect on substantia nigra injurty in 6-OHDA-lesioned rats. Brain Res. 1517, 28-35 (2013).
25. M. Kavitha, J. Nataraj, M. M. Essa, M. A. Memon, T. Manivasagam, Mangiferin attenuates MPTP induced dopaminergic neurodegeneration and improves motor impairment, redox balance and Bcl-a/Bax expression in experimental Parkinson's disease mice. Chem. Biol. Interact. 206, 239-247 (2013).
26. S. Y. Woo, J. H. Kim, M. K. Moon, et al., Discovery of vinyl sulfones as a novel class of neuroprotective agents toward Parkinson's disease therapy. J. Med. Chem., in press (2014).

What is claimed is:

1. A method of screening for therapies for a disease in a mammal comprising the steps of: (1) performing a measurement of protein-specific oxidative damage in a protein and (2) screening for a preventative therapy or a treatment of the disease that reduces the level of protein-specific oxidative damage.

2. The method of claim 1, wherein the disease is selected from the group consisting of an age-related disease, a degenerative disease, or a progeroid syndrome.

3. The method of claim 1, wherein the measurement of protein-specific oxidative damage is a phenotypic screen.

4. The method of claim 1, wherein the measurement of protein-specific oxidative damage is a measurement of protein carbonylation.

5. The method of claim 1, wherein the treatment reduces at least one intracellular reactive oxygen species.

6. The method of claim 1, wherein the treatment reduces at least one intracellular reactive nitrogen species.

7. The method of claim 1, wherein the treatment reduces the susceptibility of the protein to oxidative damage.

8. The method of any of claims 1-7, wherein the protein is α-synuclein.

9. A method of treating a disease in a mammal comprising the steps of (1) performing a diagnostic measurement of protein-specific oxidative damage in a protein and (2) providing a treatment to the mammal.

10. The method of claim 9, wherein the treatment reduces at least one intracellular reactive oxygen species.

11. The method of claim 9, wherein the treatment reduces at least one intracellular reactive nitrogen species.

12. The method of claim 9, wherein the treatment reduces protein susceptibility to oxidative damage.

13. The method of claim 9, wherein the treatment reduces at least one intracellular reactive oxygen species and reduces protein susceptibility to oxidative damage.

14. The method of claim 9, wherein the treatment reduces at least one intracellular reactive nitrogen species and reduces protein susceptibility to oxidative damage.

15. The method of claim 9, wherein the treatment reduces at least one intracellular reactive oxidative species, reduces at least one reactive nitrogen species, and reduces protein susceptibility to oxidative damage.

16. The method of claim 9, wherein the disease is selected from the group consisting of an age-related disease, a degenerative disease, or a progeroid syndrome.

17. The method of claim 9, wherein the diagnostic measurement of protein-specific oxidative damage is a phenotypic screen.

18. The method of claim 9, wherein the diagnostic measurement of protein-specific oxidative damage is a measurement of protein carbonylation.

19. The method of any of claims 10-18, wherein the protein is α-synuclein.
